(19) United States
(12) Patent Application Publication
(10) Pub. No.: US 2015/0157740 A1
(43) Pub. Date: Jun. 11, 2015

(54) METHOD FOR MANUFACTURING BIOMEDICAL SIGNAL CONDUCTIVE MEMBRANE

(71) Applicant: METAL INDUSTRIES RESEARCH & DEVELOPMENT CENTRE, Kaohsiung City (TW)

(72) Inventors: Ping-Yi Hsieh, Kaohsiung City (TW); Wei-Ching Wang, Kaohsiung City (TW); Meng-Hsiu Wu, Tainan City (TW); Wei-Te Chen, Changhua County (TW)

(21) Appl. No.: 14/504,551

(22) Filed: Oct. 2, 2014

(30) Foreign Application Priority Data
Dec. 9, 2013 (TW) 102145107

(51) Int. Cl. A61K 49/22

(52) U.S. Cl. CPC A61K 49/22

(57) ABSTRACT

A method for manufacturing a biomedical signal conductive membrane includes providing a natural polymer material, adding an acid solution for making the natural polymer material dissolved in the acid solution to form a viscous liquid mixture, performing a dry step for drying the viscous liquid mixture to form an initial stage of membrane, wherein the structure of natural polymer material within the initial stage of membrane is a straight chain, providing a cross-linking agent to perform a cross-linking step for transforming the initial stage of membrane into a gel membrane, wherein the structure of natural polymer material within the gel membrane is a cross-linked network, and performing a rinse step for removing the cross-linking agent and keeping a plurality of water molecules remaining within the cross-linked network of natural polymer material.

### FIELD OF THE INVENTION

The present invention is generally relating to a method for manufacturing a biomedical signal conductive membrane. The present invention particularly represents the method for manufacturing the biomedical signal conductive membrane that displays a particular gel membrane type that can prevents toxic monomer from generating.

### BACKGROUND OF THE INVENTION

A liquid conductive gel must be spread on a diagnostic region of subject in conventional ultrasonic diagnosis so as to make transducer probe of ultrasonic instrument moved smoothly to support the physician for detailed diagnosis. However, the liquid conductive gel will adhere to clothes or other region of subject through the movement of transducer probe therefore causing difficulty for cleaning. Besides, the liquid conductive gel is easily infected by bacteria to cause extra injury to the subjects, because the incomplete sterilization in the manufacturing process or impropriate storage. Accordingly, a newly conductive pad is developed. The newly conductive pad is produced by polymer composite, but the toxic monomer is generated during the producing process to contaminate the environment. Additionally, the human body might be injured by the toxic monomer released from the newly conductive pad. The newly conductive pad is thicker and merely adoptable to specific ultrasonic instrument, and the use of the newly conductive pad applied to the region of human is limited as well. For instance, the newly conductive pad is not able to adhere onto the joint or elbow smoothly. Therefore, the newly conductive pad can not be applied to the joint or elbow of human body.

### SUMMARY

The primary object of the present invention is to provide a method for manufacturing a biomedical signal conductive membrane. By the property modification of natural polymer material to prevent the toxic monomer generate in the manufactured process of the conductive pad.

The other object of the present invention is to provide a method for manufacturing a biomedical signal conductive membrane. A formed gel membrane has a smooth contact surface that not only prevents adherence from the gel membrane, but also apply to various regions of human body therefore increasing convenience and range of use.

A method for manufacturing a biomedical signal conductive membrane at least includes providing a natural polymer material; adding an acid solution for making the natural polymer material dissolved in the acid solution to form a viscous liquid mixture; performing a dry step for drying the viscous liquid mixture to form an initial stage of membrane, wherein the structure of natural polymer material within the initial stage of membrane is a straight chain; providing a cross-linking agent to perform a cross-linking step for transforming the initial stage of membrane into a gel membrane, wherein the structure of natural polymer material within the gel membrane is a cross-linked network; and performing a rinse step for removing the cross-linking agent and keeping a plurality of water molecules remaining within the cross-linked network of natural polymer material. The present invention adopt the natural polymer material to produce the gel membrane with cross-linking network, and the gel membrane does not generate toxic monomer that contaminates the environment and injures human body. In addition, the gel membrane can substitute for the liquid conductive gel and the conductive pad that use inconveniently, and avoids adhering to skin, transducer probe and bed sheet like the liquid conductive gel. The gel membrane can satisfy the requirement of the health care employees and subjects.

### DESCRIPTION OF THE DRAWINGS

FIG. 1 is a flow chart illustrating a method for manufacturing a biomedical signal conductive membrane in accordance with an embodiment of the present invention.

FIGS. 2A to 2D are schematic diagrams illustrating a method for manufacturing the biomedical signal conductive membrane in accordance with the embodiment of the present invention.

### DETAILED DESCRIPTION OF THE INVENTION

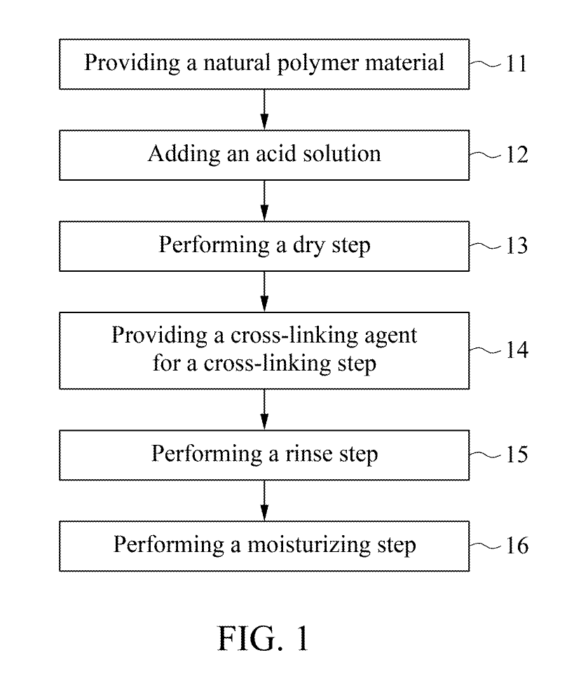

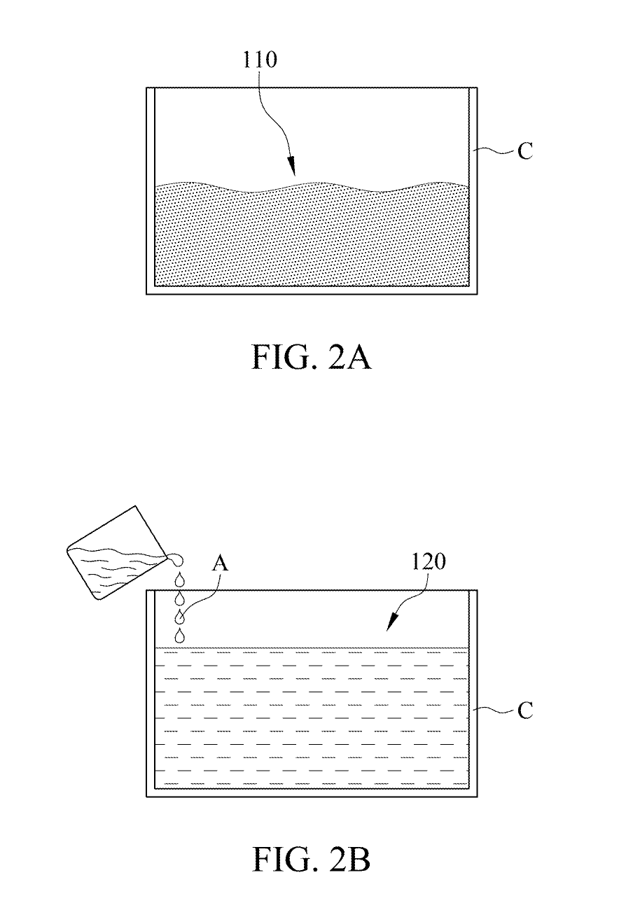

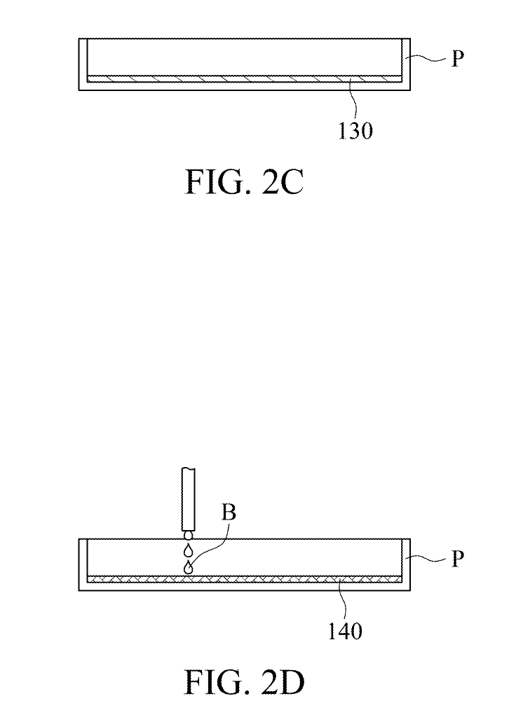

With reference to FIGS. 1 and 2A to 2D, a method for manufacturing a biomedical signal conductive membrane at least includes the following steps: first, referring to step 11 of FIG. 1 and FIG. 2A, providing a natural polymer material 110 and disposing the natural polymer material 110 into a container C such as beaker or petri dish. Preferably, the natural polymer material 110 is the polymer powder with highly hydrophilic, wherein the natural polymer material 110 is selected from the group consisting of the chitosan, alginic acid, γ-polyglutamic acid (γ-PGA) and collagen. In this embodiment, the natural polymer material 110 is selected from chitosan. The highly hydrophilic chitosan is used as a better medium for signal conduction to improve the image retrieval effect.

Next, referring to step 12 of FIG. 1 and FIG. 2B, adding an acid solution A for making the natural polymer material 110 dissolved in the acid solution A to form a viscous liquid mixture 120. In this embodiment, the acid solution A is selected from the group consisting of acetic acid, lactic acid and hydrochloride acid, and the ratio of the acid solution A to the natural polymer material 110 is 100:1. In this embodiment, 10 grams of chitosan are dissolving into 1 wt % acetic acid solution to form the viscous liquid mixture 120.

Next, referring to step 13 of FIG. 1 and FIG. 2C, disposing the viscous liquid mixture 120 into a mold (not drawn in Fig.) or a petri dish P, then placing the mold or the Petri dish P in an oven (not drawn in Fig.) to perform a dry step for drying the viscous liquid mixture 120 for 24 to 48 hours to form an initial stage of membrane 130. The structure of natural polymer material within the initial stage of membrane 130 is a straight chain.

Next, referring to step 14 of FIG. 1 and FIG. 2D, providing a cross-linking agent B to perform a cross-linking step for transforming the initial stage of membrane 130 into a gel membrane 140. In this embodiment, the thickness range of the gel membrane 140 is 100 to 2000 μm, preferably, the thickness of the gel membrane 140 is 1000 μm. The structure of natural polymer material within the gel membrane 140 is a cross-linked network. In this embodiment, the cross-linking agent B is selected from the group consisting of sodium hydroxide, calcium hydroxide and ammonium hydroxide with hydroxyl group, wherein the sodium hydroxide possesses better cross-linking effect. The cross-linking step is performed by the 1 wt % to 7 wt % cross-linking agent B for 10 to 60 minutes. After the cross-linking step, the pH value of the gel membrane 140 is pH 6.0 to pH 7.5.

After that, referring to step 15 of FIG. 1, performing a rinse step for 3 to 20 minutes for removing the cross-linking agent B and keeping a plurality of water molecules (not shown in Fig.) remaining within the cross-linked network of natural polymer material; eventually, referring to step 16 of FIG. 1, performing a moisturizing step that add a humectant agent into the gel membrane 140. In this embodiment, the humectant agent is selected from a composite moisturizing material that is composed of glycerol and hydrogel.

The present invention adopt the natural polymer material 110 to produce the gel membrane 140 that is cross-linking network, therefore, the gel membrane 140 does not generate toxic monomer that contaminates the environment and injures human body. In addition, the gel membrane 140 does not cause cytotoxicity and skin disorders. The gel membrane 140 is substitute for the liquid conductive gel and the conductive pad that use inconveniently, and avoids adhering to skin, transducer probe and bed sheet like the liquid conductive gel. The gel membrane 140 can satisfy the requirement of the health care employees and subjects. Besides, the cross-linking network of the gel membrane 140 enables to keep the water molecules remaining in the gel membrane 140 so that the gel membrane 140 possesses a certain swelling ratio. The thickness range of the gel membrane 140 is 500 to 2000 μm so as to stick on the uneven joint or elbow of human body easily therefore forming a smooth medium and solving the problem of blind spot for diagnosis via lowering the overflow of the liquid conductive gel. Preferably, the gel membrane 140 is made to be in form of cling film and possesses functions of disposability and prevention of bacteria cross-infection in a sickroom.

While this invention has been particularly illustrated and described in detail with respect to the preferred embodiments thereof, it will be clearly understood by those skilled in the art that is not limited to the specific features shown and described and various modified and changed in form and details may be made without departing from the spirit and scope of this invention.

What is claimed is:

1. A method for manufacturing a biomedical signal conductive membrane includes:
providing a natural polymer material;
adding an acid solution for making the natural polymer material dissolved in the acid solution to form a viscous liquid mixture;
performing a dry step for drying the viscous liquid mixture to form an initial stage of membrane, wherein the structure of natural polymer material within the initial stage of membrane is a straight chain;
providing a cross-linking agent to perform a cross-linking step for transforming the initial stage of membrane into a gel membrane, wherein the structure of natural polymer material within the gel membrane is a cross-linked network; and
performing a rinse step for removing the cross-linking agent and keeping a plurality of water molecules remaining within the cross-linked network of natural polymer material.

2. The method for manufacturing a biomedical signal conductive membrane in accordance with claim 1, wherein the natural polymer material is a hydrophilic polymer.

3. The method for manufacturing a biomedical signal conductive membrane in accordance with claim 1, wherein the natural polymer material is selected from the group consisting of chitosan, alginic acid, γ-polyglutamic acid (γ-PGA) and collagen.

4. The method for manufacturing a biomedical signal conductive membrane in accordance with claim 1, wherein the cross-linking agent is selected from the group consisting of sodium hydroxide, calcium hydroxide and ammonium hydroxide with hydroxyl group.

5. The method for manufacturing a biomedical signal conductive membrane in accordance with claim 1, wherein the acid solution is selected from the group consisting of acetic acid, lactic acid and hydrochloride acid.

6. The method for manufacturing a biomedical signal conductive membrane in accordance with claim 1, wherein the cross-linking step is performed for 10 to 60 minutes.

7. The method for manufacturing a biomedical signal conductive membrane in accordance with claim 1, wherein performing the dry step for 24 to 48 hours.

8. The method for manufacturing a biomedical signal conductive membrane in accordance with claim 1, wherein performing the rinse step for 3 to 20 minutes.

9. The method for manufacturing a biomedical signal conductive membrane in accordance with claim 1, wherein the thickness range of the gel membrane is 100 to 2000 μm.

10. The method for manufacturing a biomedical signal conductive membrane in accordance with claim 1 further includes a moisturizing step that after the rinse step, the moisturizing step is performing for adding a humectant agent into the gel membrane.

11. The method for manufacturing a biomedical signal conductive membrane in accordance with claim 10, wherein the humectant agent is selected from a composite moisturizing material that is composed of glycerol and hydrogel.

12. The method for manufacturing a biomedical signal conductive membrane in accordance with claim 1, wherein the concentration range of the cross-linking agent is 1 to 7 wt %.

13. The method for manufacturing a biomedical signal conductive membrane in accordance with claim 1, wherein the ratio of the acid solution to the natural polymer material is 100:1.
